SEQUENCE LISTING (29 sequences)

SEQ ID NO: 1 - DNA, 60 bp, Artificial Sequence
  synthetic oligonucleotide
tgcagctcgtgttctgtacggtgacgttaacgacgacggtaaagttaactccaccgacct

SEQ ID NO: 2 - DNA, 60 bp, Artificial Sequence
  synthetic oligonucleotide
gaccctgctgaaacgttacgttctgaaagctgtttccaccctgccgtcctccaaagctga

SEQ ID NO: 3 - DNA, 60 bp, Artificial Sequence
  synthetic oligonucleotide
aaaaaacgctgacgttaaccgtgacggtcgtgttaactcctccgacgttaccatcctgtc

SEQ ID NO: 4 - DNA, 41 bp, Artificial Sequence
  synthetic oligonucleotide
ccgttacctgatccgtgttatcgaaaaactgccgatctaac

SEQ ID NO: 5 - DNA, 60 bp, Artificial Sequence
  synthetic oligonucleotide
tgcagttagatcggcagtttttcgataacacggatcaggtaacgggacaggatggtaacg

SEQ ID NO: 6 - DNA, 60 bp, Artificial Sequence
  synthetic oligonucleotide
tcggaggagttaacacgaccgtcacggttaacgtcagcgtttttttcagctttggaggac

SEQ ID NO: 7 - DNA, 60 bp, Artificial Sequence
  synthetic oligonucleotide
ggcagggtggaaacagctttcagaacgtaacgtttcagcagggtcaggtcggtggagtta

SEQ ID NO: 8 - DNA, 41 bp, Artificial Sequence
  synthetic oligonucleotide
actttaccgtcgtcgttaacgtcaccgtacagaacacgagc

SEQ ID NO: 9 - DNA, 40 bp, Artificial Sequence
  primer
catgcaactctgcagctcgtgttctgtacggtgacgttaa

SEQ ID NO: 10 - DNA, 40 bp, Artificial Sequence
  primer
taccagatcctgcagttagatcggcagtttttcgataaca

SEQ ID NO: 11 - DNA, 60 bp, Artificial Sequence
  synthetic oligonucleotide
tgcagctcgtaaactgtacggtgacgttaacgacgacggtaaagttaactccaccgacgc

SEQ ID NO: 12 - DNA, 60 bp, Artificial Sequence
  synthetic oligonucleotide
tgttgctctgaaacgttacgttctgcgttccggtatctccatcaacaccgacaacgcgga

SEQ ID NO: 13 - DNA, 60 bp, Artificial Sequence
  synthetic oligonucleotide
cctgaacgaagacggtcgtgttaactccaccgacctgggtatcctgaaacgttacatcct

SEQ ID NO: 14 - DNA, 35 bp, Artificial Sequence
  synthetic oligonucleotide
gaaagaaatcgacaccctgccgtacaaaaactaac

SEQ ID NO: 15 - DNA, 60 bp, Artificial Sequence
  synthetic oligonucleotide
tgcagttagtttttgtacggcagggtgtcgatttctttcaggatgtaacgtttcaggata

SEQ ID NO: 16 - DNA, 60 bp, Artificial Sequence
  synthetic oligonucleotide
cccaggtcggtggagttaacacgaccgtcttcgttcaggtccgcgttgtcggtgttgatg

SEQ ID NO: 17 - DNA, 60 bp, Artificial Sequence
  synthetic oligonucleotide
gagataccggaacgcagaacgtaacgtttcagagcaacagcgtcggtggagttaacttta

SEQ ID NO: 18 - DNA, 35 bp, Artificial Sequence
  synthetic oligonucleotide
ccgtcgtcgttaacgtcaccgtacagtttacgagc

SEQ ID NO: 19 - DNA, 40 bp, Artificial Sequence
  primer
catgcatcactgcagctcgtaaactgtacggtgacgttaa

SEQ ID NO: 20 - DNA, 40 bp, Artificial Sequence
  primer
tcagacctactgcagttagtttttgtacggcagggtgtcg

SEQ ID NO: 21 - DNA, 43 bp, Artificial Sequence
  primer
cgagcgccgcgggcttgttctgtacggtgacgttaacgacgac

SEQ ID NO: 22 - DNA, 43 bp, Artificial Sequence
  primer
agccagccgcggttagatcggcagtttttcgataacacggatc

SEQ ID NO: 23 - DNA, 43 bp, Artificial Sequence
  primer
cgagcgccgcgggcttaaactgtacggtgacgttaacgacgac

SEQ ID NO: 24 - DNA, 43 bp, Artificial Sequence
  primer
agccagccgcggttagtttttgtacggcagggtgtcgatttct

SEQ ID NO: 25 - DNA, 27 bp, Artificial Sequence
  primer
gaaatacctatacatatgaaaggagtg

SEQ ID NO: 26 - DNA, 25 bp, Artificial Sequence
  primer
tggatggtataccactgaatcttac

SEQ ID NO: 27 - protein, 69 aa, Artificial Sequence
  celD dockerin domains
VLYGDVNDDGKVNSTDLTLLKRYVLKAVSTLPSSKAEKNADVNRDGRVNSSDVTILSRYLIRVIEKLPI

SEQ ID NO: 28 - protein, 67 aa, Artificial Sequence
  celS dockerin domains
KLYGDVNDDGKVNSTDAVALKRYVLRSGISINTDNADLNEDGRVNSTDLGILKRYILKEIDTLPYKN

SEQ ID NO: 29 - protein, 599 aa, Clostridium thermocellum
KLYGDVNDDGKVNSTDAVALKRYVLRSGISINTDNADLNEDGRVNSTDLGILKRYILKEIDTLPYKNPGVPSKGMANCDFVLGYDPNVLEVTEVKPGSIIKDPDPSKSFDSAIYPDRKMIVFLFAEDSGRGTYAITQDGVFATIVATVKSAAAAPITLLEVGAFADNDLVEISTTFVAGGVNLGSSVPTTQPNVPSDGVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVGNATPTKGATPTNTATPTKSATATPTRPSVPTNTPTNTPANTPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTKPPATTIPPSDDPNAIKIKVDTVNAKPGDTVNIPVRFS

(19) United States
(12) Patent Application Publication
(10) Pub. No.: US 2003/0027298 A1
(43) Pub. Date: Feb. 6, 2003

(54) Enzymatic array and process of making same

(76) Inventors: Richard R. Bott, Burlingame, CA (US); Kathleen A. Clarkson, San Francisco, CA (US); Timothy Fowler, Belmont, CA (US); Chung-Cheng Liu, San Diego, CA (US); Michael Ward, San Francisco, CA (US); Hai-Ying Xia, San Francisco, CA (US)

Correspondence Address: Genencor International, Inc., 925 Page Mill Road, Palo Alto, CA, 94034-1013, US

(21) Appl. No.: 09/955,555

(22) Filed: Sep. 17, 2001

Related U.S. Application Data
(63) Continuation of application No. 08/559,968, filed on Nov. 17, 1995, now abandoned.
(60) Provisional application No. 60/005,701, filed on Oct. 17, 1995.

(51) Int. Cl. C12N 9/00; C12N 9/02; C12N 9/24; C12N 9/50

(52) U.S. Cl. 435/183; 435/200; 435/219; 435/189

(57) ABSTRACT

An enzymatic array is provided, which composition comprises one or more enzymes non-covalently bound to a peptide backbone, wherein at least one of the enzymes is heterologous to the peptide backbone and the peptide backbone is capable of having bound thereto a plurality of enzymes. The array is useful, for example, in recovery systems, targeted multi-enzyme delivery systems, soluble substrate modification, quantification type assays, and other applications in the food industry, feed, textiles, bioconversion, pulp and paper production, plant protection and pest control, wood preservatives, topical lotions and biomass conversions.

### RELATED APPLICATIONS

This application claims the benefit of U.S. Provisional Application No. ______ (Our Docket No. GC278) filed on Oct. 17, 1995.

### BACKGROUND OF THE INVENTION

The present invention is related to an array of enzymatic activities and a process for making such an array. In particular, the present invention is related to a composition comprising at least one enzyme which is bound to a peptide backbone, wherein said backbone is capable of having bound thereto a plurality of pre-selected enzymatic activities.

Multiple enzyme aggregates have been suggested for decreasing the allergenicity of the component enzyme(s) by increasing their size. For example, PCT Publication No. 94/10191 discloses oligomeric proteins which display lower allergenicity than the monomeric parent protein and proposes several general techniques for increasing the size of the parent enzyme. Moreover, enzyme aggregates have shown improved characteristics under isolated circumstances. For example, Naka et al., Chem. Lett., vol. 8, pp. 1303-1306 (1991) discloses a horseradish peroxidase aggregate prepared by forming a block copolymer via a 2-stage block copolymerization between 2-butyl-2-oxazoline and 2-methyl-2-oxazoline. The aggregate had over 200 times more activity in water saturated chloroform than did the native enzyme.

Similarly, cross-linking of enzymes by the addition of glutaraldehyde has been suggested as a means of stabilizing enzymes. However, cross-linking often leads to losses in activity compared to native enzyme. For example, Khare et al., Biotechnol. Bioeng., vol. 35, no. 1, pp. 94-98 (1990) disclose an aggregate of E. coli β-galactosidase produced with glutaraldehyde. The enzyme aggregate, while showing improvement in thermal stability at 55° C., had an activity of only 70.8% of that of the native enzyme which was, however, considered a good retention of activity after cross-linking.

Another form of aggregated enzymes has been discovered in organisms which degrade cellulose. While cellulose is the most abundant renewable resource on earth, due to its recalcitrant nature, different microorganisms and their cellulolytic enzymes are generally required to act synergistically for the effective hydrolysis of cellulose. For example, in a plant, cellulose is commonly bound to or coated with other polymers, i.e., xylan and lignin, which hinder its degradation to sugar monomer units. Thus, a typical system will generally require a variety of enzymatic activities to effectively breakdown cellulose.

In recent years, a unique structure called the “cellulosome” has been identified as a multienzyme complex produced by various microorganisms, notably anaerobic cellulolytic bacteria of the genus Clostridium, which facilitates the breakdown of cellulose to an energy source utilizable by the microorganism in cell metabolism. The cellulosome is believed to be a discrete multifunctional, multienzyme complex which is intricately designed to maximize the cellulolytic activities within the cellulosomal complex to solubilize insoluble cellulose. Specific activities discovered within the cellulosomal complex include endo- and exo-glucanases, and hemicellulases such as xylanase.

Studies of isolated cellulosomes have elucidated a structure which is exceptionally stable but flexible enough to accommodate conformational changes during substrate interactions. The backbone of the cellulosome is believed to be a multifunctional noncatalytic polypeptide subunit which harbors the cellulose-binding function, anchors the cellulosome to the cell surface and provides a docking platform for the individual enzymatic activities. This backbone subunit, termed the scaffoldin, is the crux of the cellulosome structure.

To date, scaffolding from two different clostridial species have been described. The CipA and CipB proteins from C. thermocellum are described in Gemgross et al., Molecular Microbiology, vol. 8, no. 2, pp. 325-334 (1993) and Poole et al., FEMS Microbiol. Lett., vol. 99, pp. 181-186 (1992), respectively. The CbpA scaffoldin from C. cellulovorans and sequence is described in Shoseyov et al., Proc. Natl. Acad. Sci. USA, vol. 89, pp. 3483-3487 (1992). In the two scaffolding which have been sequenced, the majority of the domains are involved in integrating the enzymes into the complex. In both cases, a single cellulose binding domain (CBD) is present. The CBD of C. cellulovorans is the first N-terminal scaffoldin domain, whereas the C. thermocellum sequence shows a CBD in the internal domain. Sequences of CBD's from these species have been characterized by significant homology to domains of certain non-cellulosomal cellulases produced by bacteria which have been characterized as having cellulose binding activity.

Catalytic subunits of the cellulosome, made up of individual enzymatic peptides docked to the scaffoldin protein, are bound to the scaffoldin via a conserved duplicated segment which serves as a docking sequence. As reported in Wu et al., ACS Symposium Ser., Biocatalyst Design for Stability and Specificity, vol. 516, pp. 251-64 (1994) and Tokatlidis et al., FEBS Letters 10255, vol. 291, no. 2, pp.185-188 (1991), despite the lack of homology generally for each of the cellulases produced by C. thermocellum, each of the cellulase and xylanase enzymes active on the cellulosome contains a conserved, duplicated sequence of between 22-24 amino acid residues. Moreover, the CelC enzyme produced by C. thermocellum does not contain the duplicated segment and is not associated with the cellulosome.

The conserved sequence has been proposed to be a docking sequence which interacts with a complementary receptor on the scaffoldin protein, the receptor region (or internal repeating element) being reiterated nine times within the sequence of CipA and 4-6 times within the sequence of CbpA. Tokatlidis et al., Protein Engineering, vol. 6, no. 8, pp. 947-952 (1993) and Salamitou et al., J. Bacteriology, vol. 176, no.10, pp. 2822-2827 (1994), showed that a fusion protein comprising the duplicated segment of CelD from Clostridium thermocellum and the CelC endoglucanase from C. thermocellum was able to bind to the C. thermocellum CipA scaffoldin protein. It is unclear whether the activity of an enzyme incorporated into the complex is dependent on any specific attribute of the enzyme itself.

Researchers have discovered that while a cellulosome complex is generally highly efficient in degrading crystalline cellulose, enzymatic subunits (endoglucanases, exoglucanases and xylanases) dissociated from the scaffoldin protein are incapable of digesting crystalline cellulose and show activity only on amorphous or soluble cellulose. Thus, it is generally believed that the complex between the scaffoldin protein and the endoglucanases and exoglucanases is essential for the digestion of crystalline cellulose. The reason for this, however, is not clear. One hypothesis is that the cellulosome can coordinate the digestion of crystalline cellulose by interacting with the enzymatic subunits and bringing them into proximity with the fibrous substrate.

As is understood from above, considerable research has been devoted to the preparation of aggregated enzymes. However, when preparing aggregated enzymes according to these prior art teachings, it is not believed feasible to predict how certain enzymes will behave in the aggregated form. Moreover, the formation of an enzyme aggregate is an inexact science which is highly dependent on fortuity, thus presenting a significant barrier to the preparation of a multienzyme aggregate having pre-selected activities. Further, considerable research has been devoted to analyzing and understanding the cellulosomal structure. Knowledge regarding the individual components of the cellulosome and their functional interrelationships remains limited due to the complex nature of the cellulosome. Importantly, it has not been established that incorporation of heterologous enzyme components into the cellulosome complex would be successful or that such a heterologous complex could possess enough activity to be catalytically functional.

Accordingly, it would be desirable to develop a new means of preparing multiple enzyme systems useful for medical, diagnostic or industrial purposes which is capable of being customized in terms of included enzymatic activities and positional interrelationships of those enzymes so as to maximize the kinetics of the specific application. It would be further advantageous if such multiple enzyme compositions were not reliant on the existence of specific amino acids present at a specific location within each respective enzyme to allow bonding of one or several enzymes through, e.g., cross-linking, to avoid unnecessary disruption of the enzyme. Additionally, it would be advantageous to utilize the multiple enzyme structure in such a way so as to maximize the activities of the individual enzymatic activities therein. However, the prior art fails to provide a means for producing a multiple enzyme system having such characteristics.

### SUMMARY OF THE INVENTION

It is an object of the present invention to provide for a composition comprising a variety of enzymes to form a catalytic array.

It is a further object of the invention to provide for a composition comprising a variety of enzymes in the same composition, wherein the type, number and placement of the enzyme(s) within the complex may be pre-selected.

It is yet a further object of the invention to provide for a composition comprising a variety of enzymes to form a catalytic array, wherein the catalytic array allows for the performance of the enzymatic functions of the enzymes included within the array in an optimal manner.

According to the invention, a composition is provided comprising one or more enzymes non-covalently bound to a peptide backbone, wherein at least one of said enzymes is heterologous to said peptide backbone and said peptide backbone is capable of having bound thereto a plurality of enzymes.

### BRIEF DESCRIPTION OF THE DRAWINGS

FIG. 1. Amino acid sequences of celD (SEQ ID NO:27) and celS (SEQ ID NO:28) dockerin domains. Each domain contains 60-70 amino acid residues and is comprised of two homologous (but not identical) segments arranged in a linear fashion.

FIG. 2. The strategy of assembling the DNA fragment encoding the dockerin domain of celD protein. The DNA was assembled from 8 synthetic oligonucleotides through DNA ligation and DNA amplification. A PstI site was engineered at each terminus of the DNA fragment for subsequent cloning.

FIG. 3. Structure of the plasmid pAK186T15. This is a plasmid capable of replicating in E. coli and carries the resistance genes to ampicillin and chloramphenicol. The plasmid contains a promoter derived from the aprE gene of Bacillus subtilis which controls the expression of the lipase gene in Bacillus subtilis. An unique SacII site located at the COOH terminus of the lipase protein encoding sequence allows the insertion of the DNA fragment encoding the dockerin domain peptide. Once the plasmid is transformed into Bacillus subtilis, the DNA can integrate into the Bacillus chromosome at the aprE gene via the homology at the aprE promoter.

FIG. 4. Structure of plasmid pGEX-5X-3 for E. coli expression. This plasmid contains the coding sequence of glutathione-S-transferase under the control of the E. coli lac promoter. Multiple unique restriction sites were engineered immediately following the coding region of the GST protein and allow the creation of various protein fusions with GST protein. A cleavage sequence of protease Factor Xa was also engineered in the junction to allow the GST protein to be cleaved from the fusion protein.

FIG. 5. Results of binding studies showing that the complex of lipase enzyme and scaffoldin domain can be isolated through binding to cellulose when the lipase-dockerin fusion enzyme and scaffoldin having both internal repeating elements and a complete cellulose binding domain (CBD) are present in the binding reaction.

FIG. 6. The amino acid sequence of the first (1-153) and second (154-306) internal repeating units followed by the CBD (239-531) sequence. As described in Example 6, this protein was expressed in the form of GST fusion protein and was cleaved off from the GST protein moiety by the treatment of protease Factor Xa.

### DETAILED DESCRIPTION OF THE INVENTION

“Heterologous proteins” or “heterologous enzyme” means two or more proteins or enzymes which are derived from taxonomically distinct organisms. For example, a protein derived from C. thermocellum would be heterologous to a protein derived from Bacillus licheniformis.

“Catalytic array” means a multiple enzyme composition based on a peptide backbone having attached thereto a series of enzymes having at least one enzymatic activity. In a preferred embodiment, a catalytic array will include one or several enzymes the activity of which interacts together to create a synergistic effect.

“Enzyme” means a protein or peptide sequence which exhibits a specific catalytic activity toward a certain substrate or substrates. Typical enzymes for use in the present invention include protease, cellulase, lipase, peroxidase, xylanase, oxidase, esterase, oxidoreductase, laccase, lactase, lyase, polygalacturonase, β-galactosidase, glucose isomerase, β-glucoamylase, α-amylase, NADH reductase or 2,5DKG reductase.

“Non-covalent bond” or “non-covalently bound” means a molecular interaction which is not the result of a covalent bond. A non-covalent bond includes, for example, hydrophobic attraction, hydrophilic attraction, van der Waals interaction, ionic interaction or any other equivalent molecular interaction which does not involve the formation of a covalent bond.

“Peptide backbone” means a non-catalytic peptide structure which has the ability to non-covalently bind to an enzyme or protein composition.

“Scaffoldin” or “scaffolding protein” means a peptide backbone found in cellulosomal or amylosomal complexes. Specific examples of known scaffoldin proteins include the CipA or CipB proteins from C. thermocellum or the CbpA protein from C. cellulovorans. The Clostridial scaffoldin proteins are characterized by a series of internal repeating elements, or scaffoldin domains, which comprise a means for non-covalently binding thereto an enzyme. The enzyme according to the invention, thus generally includes a peptide sequence or functional region which is complementary in a bonding sense to a portion of the internal repeating element and which facilitates the non-covalent bond (a “dockerin”). The Clostridial scaffoldin proteins are further characterized by the presence of a cellulose binding domain in addition to the internal repeating element. It is contemplated as within the present invention that the scaffoldin protein would be truncated so as to eliminate or alter the cellulose binding domain. In this way, the affinity for cellulose may be modified or reduced, thus allowing for an enzyme aggregate with no or little binding capability. This arrangement may be desirable in certain applications where cellulose binding would be disadvantageous.

“Dockerin” or “docker protein” means a peptide sequence which is capable of attaching in a non-covalent manner to a peptide backbone. In a preferred embodiment, the dockerin is derived from C. thermocellum. More preferably, the dockerin is derived from the CelD and CelS dockerin from C. thermocellum. The dockerin according to the present invention is fused to an enzyme in such a way so as to facilitate non-covalent attachment of the enzyme to a peptide backbone, for example, to an internal repeating unit of a Clostridial scaffoldin protein. It is contemplated that the dockerin domain could be modified to strengthen or reduce the non-covalent bond under certain circumstances, e.g., pH, ionic strength or temperature.

“Expression vector” means a DNA construct comprising a DNA sequence which is operably linked to a suitable control sequence capable of effecting the expression of the DNA in a suitable host. Such control sequences may include a promoter to effect transcription, an optional operator sequence to control such transcription, a sequence encoding suitable ribosome-binding sites on the mRNA, and sequences which control termination of transcription and translation. Different cell types are preferably used with different expression vectors. A preferred promoter for vectors used in Bacillus subtilis is the AprE promoter, and a preferred promoter used in E. coli is the Lac promoter. The vector may be a plasmid, a phage particle, or simply a potential genomic insert. Once transformed into a suitable host, the vector may replicate and function independently of the host genome, or may, in some instances, integrate into the genome itself. In the present specification, plasmid and vector are sometimes used interchangeably. However, the invention is intended to include such other forms of expression vectors which serve equivalent functions and which are, or become, known in the art.

“Host strain” or “host cell” means a suitable host for an expression vector comprising DNA encoding the scaffoldin protein or the enzyme-dockerin protein according to the present invention. Host cells useful in the present invention are generally procaryotic or eucaryotic hosts, including any transformable microorganism in which expression can be achieved. Specifically, host strains may be Bacillus subtilis, E. coli or Trichoderma, and preferably Bacillus subtilis. Host cells are transformed or transfected with vectors constructed using recombinant DNA techniques. Such transformed host cells are capable of both replicating vectors encoding the peptide backbone, scaffoldin or enzyme-dockerin fusion and its variants (mutants) or expressing the desired peptide product.

“Derivative” means a DNA or amino acid sequence which has been modified from its progenitor or parent sequence, through either biochemical,. genetic or chemical means, to effect the substitution, deletion or insertion of one or more nucleotides or amino acids, respectively. A “derivative” within the scope of this definition will retain generally the properties or activity observed in the native or parent form to the extent that the derivative is useful for similar purposes as the native or parent form.

The present invention includes a composition comprising one or more enzymes non-covalently bound to a peptide backbone, wherein at least one enzyme is derived from an organism heterologous to the peptide backbone and the peptide backbone is capable of having bound thereto a plurality of enzymes. In a preferred embodiment, the peptide backbone is derived from the CipA or CipB proteins of C. thermocellum or the CbpA protein of C. cellulovorans.

The non-covalently bound enzyme can be any enzyme having a particular desired enzymatic activity. Suitable enzymes include protease, cellulase, lipase, peroxidase, xylanase, oxidase, esterase, oxidoreductase, laccase, lactase, lyase, polygalacturonase, β-galactosidase, glucose isomerase, β-glucoamylase, α-amylase, NADH reductase or 2,5DKG reductase. However, any enzyme or protein may be utilized according to the present invention.

The enzyme preferably is genetically engineered so that in its expressed form it comprises a fusion protein which includes a catalytically active portion of the enzyme and an amino acid sequence which corresponds to a dockerin and which is complementary to a portion of the peptide backbone. Such complementarity is possible where the peptide backbone is derived from the scaffoldin protein produced by bacterial species such as Clostridium sp. and the dockerin protein which is fused to the enzyme is derived from the same species. Moreover, it is believed that the dockerin and scaffoldin proteins derived from the various Clostridium species, e.g., Clostridium thermocellum and Clostridium cellulovorans contain significant homology. Accordingly, it is contemplated as within the scope of the present invention to provide for a dockerin protein from Clostridium thermocellum and a scaffoldin protein derived from Clostridium cellulovorans, or vice versa. According to this embodiment, the enzyme-dockerin fusion will “dock” or non-covalently bind to an internal repeating element within the scaffoldin protein for which the dockerin is complementary.

Especially preferred are the dockerins derived from C. thermocellum and C. cellulovorans, for example the dockerin segment of the CelD or CelS proteins which are produced by C. thermocellum. Because the CelD or CelS dockerin segment is believed to be complementary to the internal repeating elements of, C. thermocellum, the fusion protein comprising the CelD or CelS dockerin and the desired enzyme activity will dock to the scaffoldin derived from C. thermocellum.

The present invention includes a catalytic array wherein more than one enzyme, at least one of which is heterologous to the peptide backbone or the dockerin segment, is non-covalently bound to the peptide backbone. In this embodiment, it is possible to manipulate the conditions of the reaction to ensure that the catalytic array comprises a variety of enzymatic activities. Examples of such an array could include a cellulase and a xylanase for use in hydrolyzing lignocellulosic material or a combination of a protease, an amylase, a cellulase and a lipase for use in detergents. In such a way it would be possible to introduce several enzymatic activities into an array which are relevant to a particular application.

Several strategies can be utilized for the production of multiple enzyme arrays according to the present invention. For example, Applicants believe that different dockerins will preferentially bind to specific internal repeating units within the scaffoldin. To take advantage of this preferential binding, a first fusion enzyme-dockerin should be prepared in which the dockerin is specific for a first internal repeating element, and a second fusion enzyme-dockerin should be prepared in which the dockerin is specific for a second internal repeating element. When the two fusion enzymes are bound to scaffoldin, which either in a natural state or after genetic manipulation has a preselected arrangement of internal repeating elements, the first fusion enzyme will bind to the first internal repeating element and the second fusion enzyme will bind to the second internal repeating element. This procedure can be repeated for a plurality of different enzyme-dockerin fusions and internal repeating elements to create a reproducible enzymatic array. As another example, two different enzymes or proteins could bind to each other by creating one enzyme fusion with a dockerin domain and another enzyme fusion with an internal repeating unit derived from the scaffoldin. When these two enzyme fusions are mixed, a complex would be formed due to the interaction of the dockerin and the internal repeating unit. Conventional protein purification techniques may also be used to purify partial complexes when a plurality of different enzyme-dockerin fusions are binding to multiple internal repeating elements and preferential interactions can not be satisfactorily employed.

The present invention may find further use in reducing allergenicity, producing synergistic effects, facilitating selective modification of substrate (i.e., a large complex would be unable to penetrate the pores of cellulose or other substrates ensuring that activity is limited to the surface of the substrate), by taking advantage of the cellulose binding domain feature of the present invention the complex would be capable of being immobilized for chromatographic separations or for soluble substrate modification. The present invention could also find advantage in recovery systems. For example, by adding the scaffoldin domain, it would be possible to recover enzymes after completion of an application. Similarly, by adding an appropriate amount of scaffoldin domain, it would be possible to quantify the amount of enzyme in solution in a manner similar to an antibody/antigen type assay, i.e., after addition of the scaffoldin and removal of the enzyme complex, the difference in activity could be measured.

Additionally, a targeted multi-enzyme delivery system is enabled by the present invention. For example, a drug delivery system which releases enzyme under certain conditions which effect the non-covalent bond, e.g., temperature, pH or ionic strength, which are known to exist in a specific physiological environment. Such delivery systems would also be useful in, for example, the food industry/processing, animal feed, textiles, bioconversion, pulp and paper production, plant protection and pest control, as a wood preservative, topical lotions, and biomass conversions.

Several advantages are provided for by the present invention over the prior art method of simply adding enzymes individually to a system. For example, an advantage of the present invention is that the protein will have significantly less allergenicity due to its large size; an enzyme which is part of the array would be capable of acting as a substrate receptor for the other enzymes; non-proteolytic enzymes would be more resistant to proteolytic attack when present in a larger complex; different enzymes working together within a limited diffusion sphere would be expected to render a substrate more accessible to each other; and complexes would assure that desired stoichiometry and mixing characteristics are present.

Additionally, an advantage of the present invention is that by introducing a precise orientation to the array, it will be possible to optimize reactions when more than one enzymatic action is necessary to accomplish a specific goal. In this way, it should be possible to optimize a multi-enzyme system in such a way that the multi-enzyme array has superior characteristics in comparison with individual combined enzymes in solution in terms of allergenicity, activity, selectivity or stability.

An example of a system which would benefit from the instant invention is the degradation of lignocellulosic materials which have interlocking bonds between cellulose polymers and xylan in the matrix. By combining cellulase and xylanase according to the present invention, it may be possible to produce a catalytic array which has a synergistic effect on degrading the complex structure of wood. While the native cellulosomal structure is believed to include cellulolytic activity and xylanolytic activity, the present invention allows the optimization of the system by using more efficient cellulolytic enzymes or combinations of enzymes than those derived from the species which produces the cellulosome.

Another example of such a system is the combination of a lipase, an amylase and a protease in a laundry detergent. By incorporating such an array in a detergent, it would be possible to more efficiently remove complex stains, e.g., food stains, which may include a matrix of fats, starches and proteins.

Yet another example of such a system would be the inclusion of several enzymes which are necessary for carrying out a particular series of steps in a metabolic pathway. For example, in the reduction of 2,5-diketo-D-gluconic acid to 2-keto-L-gulonic acid, it would be desirable to include both the E4 enzyme which catalyzes this reaction and an enzyme which facilitates necessary cofactor regeneration, i.e., an NADP reductase enzyme which will satisfy the requirement of E4 for NADPH to effect catalysis. By including both the E4 enzyme and the NADP reductase enzyme in close proximity via a catalytic array, the kinetics of the reaction catalyzed by E4 should be improved.

### EXAMPLES

### Example 1

### Cloning of DNA Encoding the CelD Dockerin

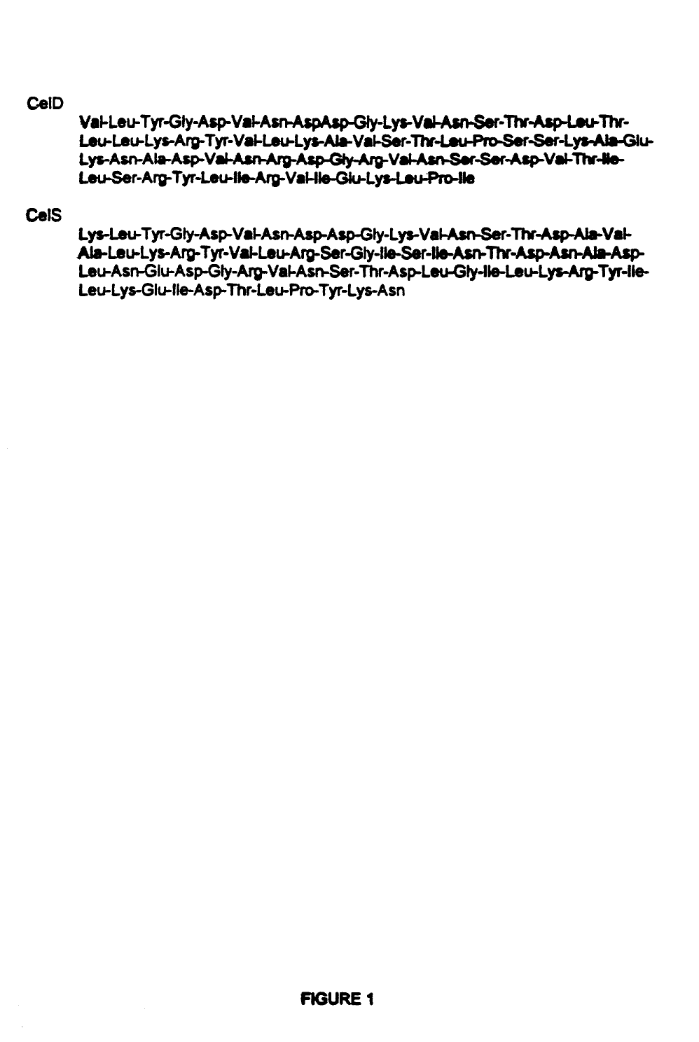

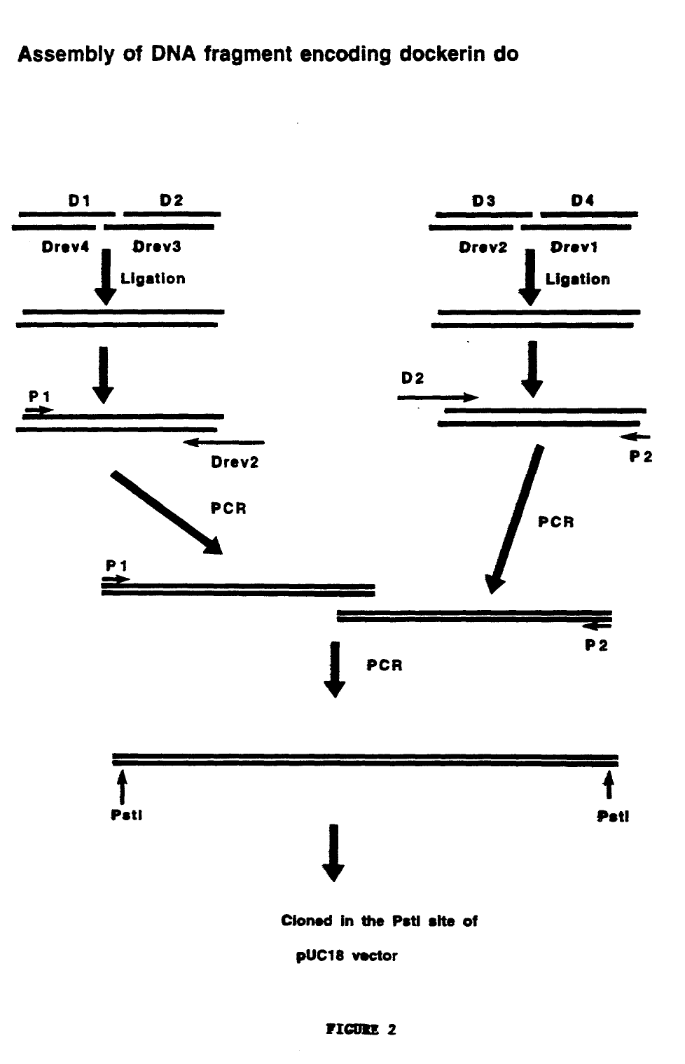

DNA encoding dockerin was constructed by assembling synthetic DNA fragments and cloning the assembled fragment in a conventional cloning vector. A scheme for this strategy is shown in FIG. 2. A total of 8 synthetic DNA fragments were synthesized, D1-D4 and Drev1-Drev4. These oligos were in the range of 60 residues and contained overlapping encoding sequence of the CelD dockerin domain The amino acid sequence of the CelD dockerin domain is shown in FIG. 1. The nucleotide sequence of the synthetic DNA used is as shown below. Primer1 and Primer2 are two primers used to amplify one DNA fragment in PCR.

| | |
|---|---|
| D1 | |
| 5′TGCAGCTCGTGTTCTGTACGGTGACGTTAACGACGACGGTAAAGTTAACTCCACCGACCT3′ | (SEQ ID NO:1) |
| D2 | |
| 5′GACCCTGCTGAAACGTTACGTTCTGAAAGCTGTTTCCACCCTGCCGTCCTCCAAAGCTGA3′ | (SEQ ID NO:2) |
| D3 | |
| 5′AAAAAACGCTGACGTTAACCGTGACGGTCGTGTTAACTCCTCCGACGTTACCATCCTGTC3′ | (SEQ ID NO:3) |
| D4 | |
| 5′CCGTTACCTGATCCGTGTTATCGAAAAACTGCCGATCTAAC3′ | (SEQ ID NO:4) |
| Drev1 | |
| 5′TGCAGTTAGATCGGCAGTTTTTCGATAACACGGATCAGGTAACGGGACAGGATGGTAACG3′ | (SEQ ID NO:5) |
| Drev2 | |
| 5′TCGGAGGAGTTAACACGACCGTCACGGTTAACGTCAGCGTTTTTTTCAGCTTTGGAGGAC3′ | (SEQ ID NO:6) |
| Drev3 | |
| 5′GGCAGGGTGGAAACAGCTTTCAGAACGTAACGTTTCAGCAGGGTCAGGTCGGTGGAGTTA3′ | (SEQ ID NO:7) |
| Drev4 | |
| 5′ACTTTACCGTCGTCGTTAACGTCACCGTACAGAACACGAGC3′ | (SEQ ID NO:8) |
| Primer1 | |
| 5′CATGCAACTCTGCAGCTCGTGTTCTGTACGGTGACGTTAA3′ | (SEQ ID NO:9) |
| Primer2 | |
| 5′TACCAGATCCTGCAGTTAGATCGGCAGTTTTTCGATAACA3′ | (SEQ ID NO:10) |

The fragments were assembled by using a combination of DNA ligation and polymerase chain reaction (PCR) techniques. The dockerin domain CelD has two homologous 30 amino acid regions. Assembly of the first half sequence of CelD dockerin domain was constructed by ligating the mixture of oligos D1, D2, Drev3 and Drev4. The ligated DNA was then amplified by PCR reaction using Drev2 and Primer1 as primers. In a separate reaction, the second half of the CelD dockerin domain was similarly constructed by ligating the mixture of oligos D3, D4, Drev1 and Drev2 and amplified by PCR using D2 and Primer2 as primers. PCR was performed in a Perkin Elmer thermocycler using a program consisting of 30 cycles of [95° C. for 10 seconds, 42° C. for 15 seconds, 65° C. for 30 seconds] followed by incubating at 95° C. for 10 seconds and 72° C. for 5 minutes. The DNA product of both PCR reactions was purified away from the unused primer with the QIAquick spin PCR purification kit (QIAGEN, CA).

The assembly reaction to construct the DNA encoding the entire CelD dockerin peptide sequence was by PCR. Both DNA fragments obtained in the procedure described above were mixed with Primer1 and PrimeR and a PCR was carried out under the same conditions as described above. Unused primers were again removed from the PCR product by a QIAquick spin PCR purification kit.

To clone the amplified DNA product, the DNA was first digested with the restriction enzyme PstI (Boehringer Mannheim Biochemicals, IN.) and run on a 1% low melting point agarose gel. A DNA fragment with the size of 220 base-pairs (bp) was purified from the gel by using a QIAquick gel extraction kit (QIAGEN INC., CA). The purified fragment was ligated into PstI digested pUC18 plasmid DNA (New England Biolabs, MA), transformed into E. coli JM101, and plated on agar plates having 50 μg/ml carbenicillin and 0.004% X-gal as a selectable marker. The white colonies from the agar plates were inoculated into 5 ml LB medium containing 50 pg/ml carbenicillin. Plasmid DNA was extracted from the cell by using a QIAprep spin plasmid kit (QIAGEN INC., CA) and digested with restriction enzyme PstI. The plasmid DNA which contained the expected PstI fragment insert (about 220 bp) was analyzed and verified by DNA sequencing (ABI 373A DNA Sequencer, Applied Biosystems, CA).

A DNA encoding the CelS dockerin was constructed by using the same procedure as that for CelD and similarly verified by DNA sequencing. The DNA fragments used to construct CelS encoding DNA and the DNA primers used in PCR are:

| | |
|---|---|
| S1 | |
| 5′TGCAGCTCGTAAACTGTACGGTGACGTTAACGACGACGGTAAAGTTAACTCCACCGACGC3′ | (SEQ ID NO:11) |
| S2 | |
| 5′TGTTGCTCTGAAACGTTACGTTCTGCGTTCCGGTATCTCCATCAACACCGACAACGCGGA3′ | (SEQ ID NO:12) |
| S3 | |
| 5′CCTGAACGAAGACGGTCGTGTTAACTCCACCGACCTGGGTATCCTGAAACGTTACATCCT3′ | (SEQ ID NO:13) |
| S4 | |
| 5′GAAAGAAATCGACACCCTGCCGTACAAAAACTAAC3′ | (SEQ ID NO:14) |
| Srev1 | |
| 5′TGCAGTTAGTTTTTGTACGGCAGGGTGTCGATTTCTTTCAGGATGTAACGTTTCAGGATA3′ | (SEQ ID NO:15) |
| Srev2 | |
| 5′CCCAGGTCGGTGGAGTTAACACGACCGTCTTCGTTCAGGTCCGCGTTGTCGGTGTTGATG3′ | (SEQ ID NO:16) |
| Srev3 | |
| 5′GAGATACCGGAACGCAGAACGTAACGTTTCAGAGCAACAGCGTCGGTGGAGTTAACTTTA3′ | (SEQ ID NO:17) |
| Srev4 | |
| 5′CCGTCGTCGTTAACGTCACCGTACAGTTTACGAGC3′ | (SEQ ID NO:18) |
| Primer3 | |
| 5′CATGCATCACTGCAGCTCGTAAACTGTACGGTGACGTTAA3′ | (SEQ ID NO:19) |
| Primer4 | |
| 5′TCAGACCTACTGCAGTTAGTTTTTGTACGGCAGGGTGTCG3′ | (SEQ ID NO:20) |

### Example 2

### Construction of DNA Encoding Protein Fusions of Pseudomonas mendocino Lipase and Dockerin Domains from CelD and CelS

The recombinant gene encoding the lipase of Pseudomonas mendocino contains an unique SacII site at the COOH terminus of the coding region. To fuse the lipase gene with CelD dockerin domain at this SacII site, a SacII recognition sites was created at DNA encoding CelD and CelS dockerin domains. To this end, a PstI digested CelD fragment (from pUC18 plasmid described in Example 1) was used as a template in the PCR reaction with the following two primers:

| | |
|---|---|
| D-SacII | |
| 5′CGAGCGCCGCGGGCTTGTTCTGTACGGTGACGTTAACGACGAC3′ | (SEQ ID NO:21) |
| revD-SacII | |
| 5′AGCCAGCCGCGGTTAGATCGGCAGTTTTTCGATAACACGGATC3′ | (SEQ ID NO:22) |

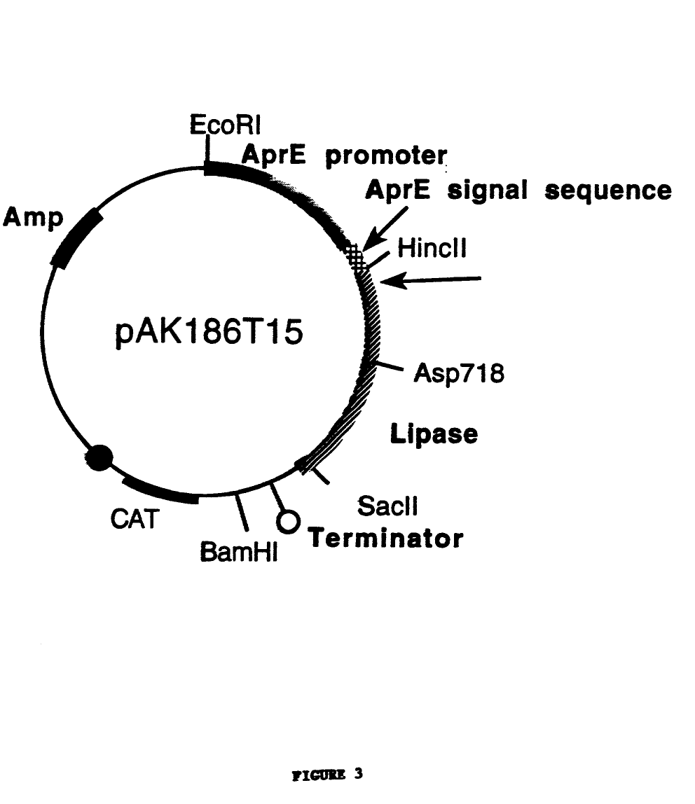

After the PCR reaction, the amplified DNA was purified away from the unincorporated primers and digested with restriction enzyme SacIII. The SacII digested DNA fragment was then cloned into SacII digested pAK186T15 plasmid (FIG. 3). pAK186T15 is a recombinant plasmid designed to express the Pseudomonas lipase gene in Bacillus subtilis and a correct insertion of the CelD encoding sequence at the SacII site will create a coding sequence for a lipase-CelD fusion protein and, therefore, the expression of lipase-CelD dockerin domain fusion protein in Bacillus subtilis. The DNA sequence of the obtained recombinant DNA was verified by sequencing.

The DNA fragment encoding CelS was cloned in a similar fashion into the pAK186T15 plasmid to create a recombinant plasmid capable of directing the expression of lipase-CelS fusion protein in Bacillus subtilis. The primers used in the PCR for obtaining SacII containing fragments encoding CelS dockerin domain are:

| | |
|---|---|
| S-SacII | |
| 5′CGAGCGCCGCGGGCTTAAACTGTACGGTGACGTTAACGACGAC3′ | (SEQ ID NO:23) |
| revS-SacII | |
| 5′AGCCAGCCGCGGTTAGTTTTTGTACGGCAGGGTGTCGATTTCT3′ | (SEQ ID NO:24) |

### Example 3

### Transformation of Recombinant Plasmids into Bacillus subtilis BG3755 and the Production of Lipase-Dockerin Fusion Protein

Bacillus subtilis BG3755 was inoculated into 2.5 ml of 1 × MG (1× Bacillus salts, 0.5% glucose, and 5 mM MgSO4) with 0.1 mg/ml amino acid mixture, and incubated with shaking at 37° C., 250 rpm for 5.5 hours. 150 μl of the growing cells were added into 1 ml of 1× MG containing 0.01% CAA. After incubation, 200 μl of the medium was transferred to another glass tube with about 2 μg plasmid DNA, and incubated with shaking at 37° C., 170-200 rpm for approximately 1.5 hours. The culture was then plated on LB plates containing 5 μg/ml chloramphenicol. The chloramphenicol-resistant colony represents cells in which at least one copy of the PAK186T15 is integrated into the chromosome.

To achieve a higher level of expression, the culture was selected for resistance to a higher level of chloramphenicol to obtain cells with more copies of the PAK186T15 integrated into the chromosome. To do this, a colony of BG3755 from the plate with 5 μg/ml chloramphenicol was inoculated in 10 μg/ml chloramphenicol-containing LB medium and grown at 200 rpm overnight. The overnight culture was diluted (1:100) to LB medium with 25 μg/ml chloramphenicol and incubated with shaking at 37° C. for another 4 hours. 50 μl of the culture was then plated on the LB plate with 25 μg/ml chloramphenicol, and incubated at 37° C. overnight. Resistant colonies represented cells with several copies of PAK186T15 integrated in the chromosome.

For the expression of lipase-CelD or lipase-CelS fusion proteins, one colony from the plate containing 25 μg/ml chloramphenicol was inoculated into 5 ml LB medium with 25 μg/ml chloramphenicol and 1% glycerol, and incubated with shaking at 30° C. overnight. Overnight culture was diluted 1:25 into a shake flask medium comprising 0.03 g MgSO₄, 0.22 g K₂HPO₄, 11.3 g Na₂HPO₄, 6.1 g NaH₂PO₄.H₂O, 3.6 g urea, 350 g Maltrin M150, 210 g glucose and 7.0 g soy flour per 1 liter of H₂O, and incubated with shaking at 200-225 rpm for 48 hours. The level of expression was determined by assaying the enzymatic activity of lipase.

### Example 4

### Assay of Lipase Activity

Lipase activity was determined by the hydrolysis of a colorimetric substrate. After fermentation, the culture suspension was centrifuged at 12,000 rpm for 30 minutes to remove cells and cell debris and the supernatant was collected. The collected supernatant was diluted (1:10-20) with lipase buffer (50 mM Tris-HCl, pH 7.5, 0.02% Triton X-100). 10 μl of the diluted sample and 10 μl of the lipase substrate, p-nitrophenyl butyrate (PNB), were added to 980 μl of pre-warmed (25° C.) lipase buffer. A preset program (measure for 1 second, every 2 seconds for 14 seconds at 410 nm) was run in a 8451A DIODE ARRAY Spectrophotometer to obtain the reaction rate. The lipase activity (μg/ml) was derived from the reaction rate multiplied by a conversion factor of 0.06 and dilution factor. The linear range of lipase activity in this assay is 30-120 μg/ml.

### Example 5

### Cloning of DNA Encoding Scaffoldin from C. thermocellum

DNA sequences of the gene encoding the entire CipA protein which were utilized in this Example are described in Gemgross et al., Molecular Microbiology, vol. 8, no. 2, pp. 325-334 (1993). DNA encoding an individual scaffoldin domain such as IRE1, IRE2, etc., or any combinations of its sequential repeat (Gerngross, supra) can be obtained by PCR with appropriate primers and C. thermocellum chromosomal DNA as a template. To prepare chromosomal DNA, C. thermocellum was grown at 60° C. under anaerobic condition. Chromosomal DNA was isolated by following the procedure “Preparation of Genomic DNA from Bacteria” described in Current Protocols in Molecular Biology (John Wiley & Sons, Inc., 1995).

Different primer combinations can be used to amplify different parts of the CipA gene. For example, to amplify and clone the DNA encoding the first (IRE1), second (IRE2) and the cellulose binding domain (CBD) of the CipA protein, the following primers were used:

| | |
|---|---|
| IRE1/IRE2 | |
| 5′GAAATACCTATACATATGAAAGGAGTG3′ | (SEQ ID NO:25) |
| CBDrev | |
| 5′TGGATGGTATACCACTGAATCTTAC3′ | (SEQ ID NO:26) |

The extracted chromosomal DNA from C. thermocellum and the primers described above were amplified by PCR reaction (30 cycles of [95° C. for 10 seconds, 42° C. for 30 seconds, and 65° C. for 30 seconds], followed by incubating at 95° C. for 10 seconds and 72° C. for 5 minutes). The amplified DNA was ligated into the TA cloning vector PCRII (from Invitrogen, CA). One Shot™ INV aF′ competent cells (from Invitrogen, CA) were transformed with the ligation mixture under the conditions recommended by the manufacturer. Six colonies were inoculated and the extracted DNA was digested with restriction enzymes EcoRI and HindIII respectively for examining the size of the DNA insert and the orientation. Plasmids containing DNA inserts with expected size and restriction pattern were further analyzed by DNA sequencing. Clones which contained the correct insert (IRE1+IRE2+CBD) were identified. One clone was found to contain DNA encoding IRE1+IRE2 followed by only 60% of CBD.

### Example 6

The Expression of the Scaffoldin as GST-Scaffoldin Fusion Proteins

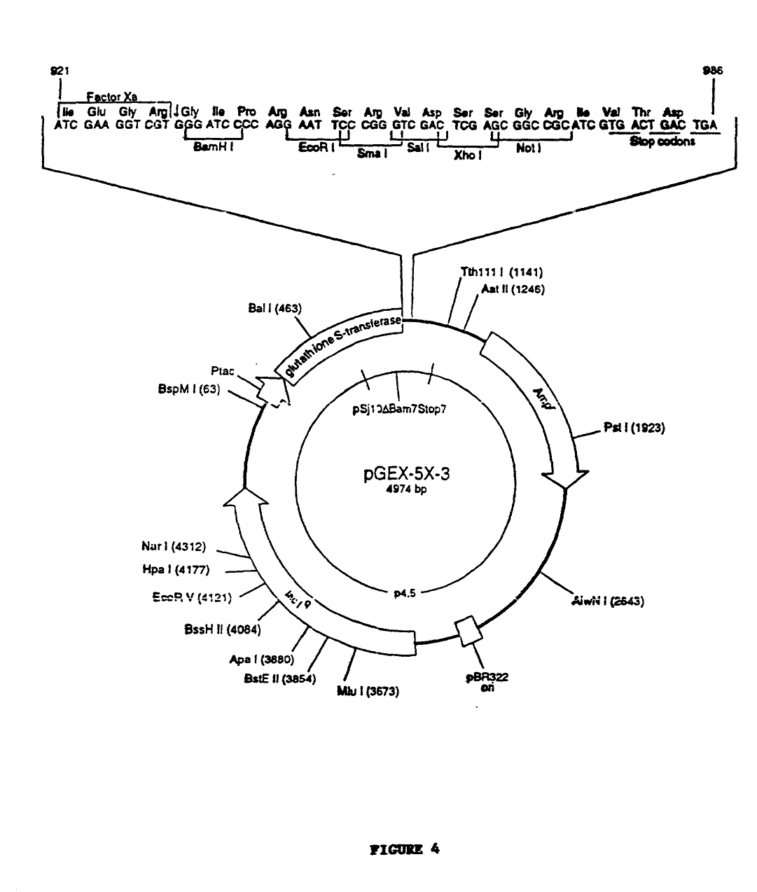

Expression of fusion protein with GST (Glutathione-S-Transferase) was performed in E. coli. The scaffoldin GST fusion protein can be conveniently recovered from cell extract by the affinity of GST protein moiety toward glutathione column. To produce the scaffoldin GST fusion, DNA encoding the first two repeating domains and 60% of cellulose binding domain (CBD) (IRE1+IRE2+60%CBD) at its forward orientation was isolated from the clones of Example 5 and digested with restriction enzyme SpeI (5′-ACTAGT-3′) and the 5′ overhangs filled in by T4 DNA polymerase in the presence of dNTP's. The DNA was then digested with NotI to release the DNA insert as a blunt end-NotI fragment and subcloned into the expression vector PGEX-5X-3 (Pharmacia Biotech, NJ) cleaved with SmaI and NotI (blunt end and NotI-containing vector). A diagram showing the restriction pattern and the multiple sites used in making the GST protein fusion is shown in FIG. 4. The resultant recombinant will contain the coding DNA for a GST fusion protein with scaffoldin domain (IRE1+IRE2+60%CBD) fused with GST protein at the COOH terminus of the GST protein. To create the gene encoding the first two repeating domains of the CipA protein and the full length of the CBD fused to the COOH-terminus of the GST protein, a clone containing a DNA insert in the proper orientation encoding IRE1+IRE2+CBD was digested with HindIII. The HindIII fragment containing the last part of the CBD (about 420 bp) was isolated and subcloned into the HindIII cleaved PGEX-5X-3 (FIG. 4) DNA containing IRE1+IRE2+60%CBD (from above) to restore the complete coding region of the CBD. E. coli 294 competent cells were used in this transformation and KpnI digestion was used to verify the insertion and the correct orientation of the HindIII insert.

For the expression of GST fusion proteins, the clone which contained PGEX-5X-3 with the desired scaffoldin-GST fusion was inoculated into 5 ml LB medium with 50 μg/ml carbenicillin, and incubated by shaking at 37° C overnight. The overnight culture was diluted 1:50 into fresh LB medium supplemented with 50 pg/ml carbenicillin. The cells were grown at 37° C. to mid-log phase (A600=0.6-1.0). The expression of fusion proteins was induced by adding isopropyl-b-D-thiogalactoside (IPTG) to a final concentration of 1.0 mM. The cells were grown for an additional 3 hours at 37° C. after the addition of IPTG and the cell pellets were harvested by centrifugation.

### Example 7

### Isolation of GST-Scaffoldin Fusion Protein from E. coli

The E. coli cell pellets (from Example 6) were resuspended in buffer A (50 mM Tris-HCl pH 7.5, 1 mM EDTA, 5% glycerol, 1 mM PMSF) at a concentration of 20 OD₆₀₀. Cells were lysed by sonication and the cell lysate was cleared from cell debris by centrifugation. The clarified supernatant after centrifugation was loaded onto a glutathione sepharose column equilibrated with buffer A. GST-scaffoldin fusion proteins were eluted out with elution buffer (50 mM Tris-HCl pH 8.0, 10 mM glutathione reduced form) after the column was washed with 50 mM Tris-HCl, pH 8.0. The size of the purified GST-scaffoldin fusion protein can be verified by comparing the apparent molecular weight, determined by running on a 10% SDS-PAGE gel, with that deduced from the protein sequence. The fusion protein contains a peptide sequence, IEGR, at the junction of the GST protein and the scaffoldin domain. The structure of the fusion protein can be further characterized by the sensitivity of the fusion protein to a specific protease, Factor Xa. Cleavage by Factor Xa can also be used to separate the GST protein from the scaffoldin domain. For the cleavage of fusion protein with Factor Xa, the following conditions were used: Factor Xa concentration, 1% (w/w) of fusion protein; reaction buffer, 50 mM Tris-HCl pH 7.5, 150 mM NaCl, 1 mM CaCl₂; incubation temperature, 14° C.; incubation time, 14-16 hours. After cleavage, two protein species with molecular weight corresponding to GST and scaffoldin domain were detected on the SDS-PAGE gel followed by commassie blue staining.

### Example 8

### The Binding of Lipase-Dockerin Fusion Protein to Scaffoldin Protein

Lipase-dockerin domain fusion protein expressed in the crude fermentation broth (from Example 3) was concentrated by Centriprep 10 (Amicom, Inc., MA) and then dialyzed against 100-500 volumes of TBS (10 mM Tris-HCl pH 7.5, 0.9% NaCl) to remove phosphate from the shake flask medium. The dialyzed lipase-dockerin domain fusion protein was used directly in the binding assay without further purification.

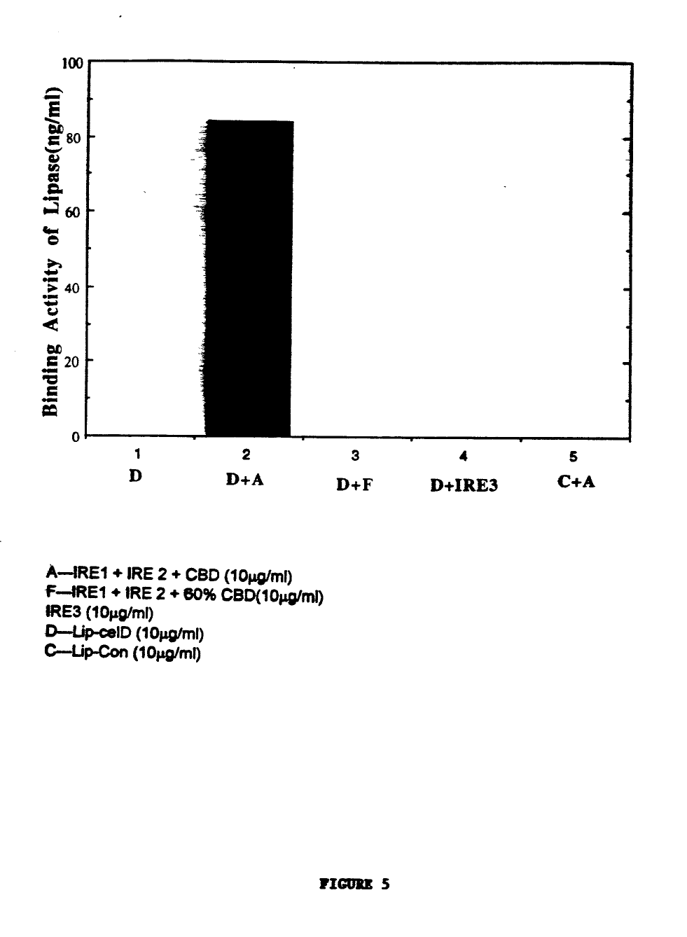

The binding assay was performed by incubating scaffoldin protein containing IRE1+IRE2+CBD (about 4 μg/ml) with lipase-dockerin domain fusion protein (about 20 μg/ml) in a total volume of 0.5 ml at room temperature for 2 hours in a buffer containing 1 mM CaCl₂. 10 mg of Avicel (cellulose) was added to the mixture and incubated for another 1 hour at room temperature. The cellulose was retained by filtering and followed by washing. The retained cellulose was resuspended in 1 ml of the lipase assay buffer and assayed for lipase activity by colorimetric assay (same as Example 4). The amount of lipase activity detected in the retained fraction represents the amount of lipase which is binding to scaffoldin protein which in turn was retained by the cellulose through the CBD. Control experiments were run by incubating truncated scaffoldin having a partial CBD (IRE1+IRE2+60%CBD) (expected to be inactive in binding to Avicel) with lipase-CelD fusion, scaffoldin domain (IRE3) in the absence of CBD with lipase-CelD fusion and scaffoldin protein containing IRE1+IRE2+CBD with lipase protein not having a dockerin domain. As can be seen in FIG. 5, significant binding of lipase to the cellulose was observed only when both the scaffoldin with intact CBD and dockerin domain were present in the incubation.

Of course, it should be understood that a wide range of changes and modifications can be made to the preferred embodiments described above. It is therefore intended to be understood that it is the following claims, including all equivalents, which define the scope of the invention.

We claim:

1. A composition comprising one or more enzymes non-covalently bound to a peptide backbone, wherein at least one of said enzymes is heterologous to said peptide backbone and said peptide backbone is capable of having bound thereto a plurality of enzymes.

2. The composition according to claim 1, comprising at least two enzymes non-covalently bound to said peptide backbone.

3. The composition according to claim 1, wherein said one or more enzyme comprises protease, cellulase, lipase, peroxidase, xylanase, oxidase, esterase, oxidoreductase, laccase, lactase, lyase, polygalacturonase, β-galactosidase, glucose isomerase, β-glucoamylase, β-amylase, NADH reductase or 2,5DKG reductase.

4. The composition according to claim 1, wherein said peptide backbone comprises scaffoldin derived from a microorganism which produces a cellulosomal or amylosomal complex.

5. The composition according to claim 4, wherein said scaffoldin is derived from Clostridium sp. and comprises at least one internal repeating element and at least one cellulose binding domain.

6. The composition according to claim 1, wherein said enzyme is non-covalently bound to said peptide backbone by means of a dockerin region of said enzyme.

7. The composition according to claim 6 wherein said dockerin region comprises a dockerin derived from Clostridium sp. or a derivative thereof capable of non-covalently binding to said peptide backbone.

8. The composition according to claim 6 wherein said dockerin comprises an amino acid sequence according to the sequence of CelS or CelD as shown in FIG. 1.

9. The composition according to claim 5, wherein said scaffoldin comprises CipA, CipB or CbpA.

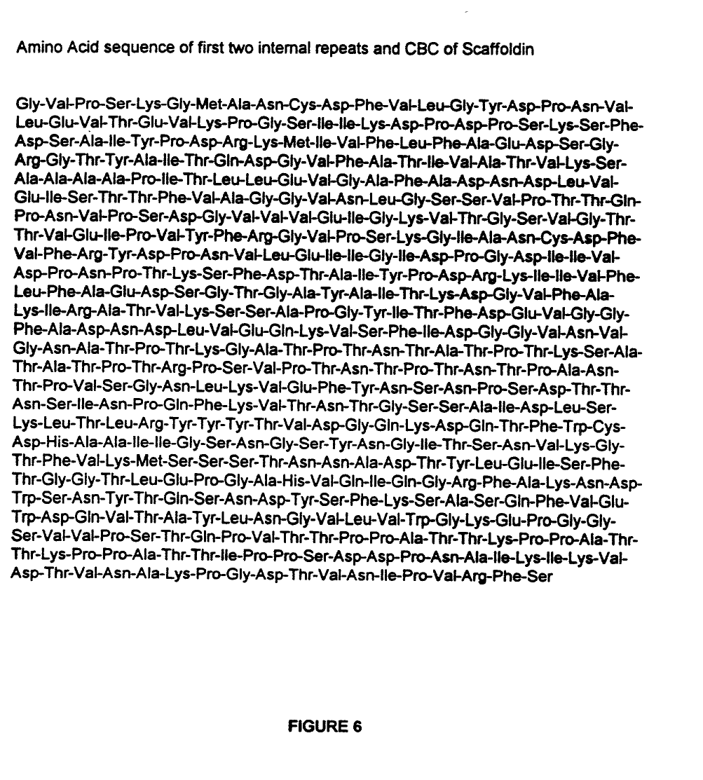

10. The composition according to claim 9, wherein said scaffoldin comprises an amino acid sequence described in FIG. 6.

11. The composition according to claim 5, wherein said scaffoldin comprises a plurality of internal repeating elements.

12. The composition according to claim 11, wherein said heterologous enzyme further comprises a dockerin region capable of binding with said scaffoldin protein.

13. The composition according to claim 12, wherein said dockerin region forms a non-covalent bond with said internal repeating units.

14. A composition comprising a scaffoldin protein bound to a heterologous enzyme.

15. A composition comprising an array of enzymes bound to a peptide backbone, wherein said composition is produced by a process comprising:
(a) expressing DNA encoding said peptide backbone in a microorganism having been transformed with DNA encoding said peptide backbone;
(b) expressing DNA encoding said enzyme in a microorganism having been transformed with DNA encoding said enzyme; and
(c) binding said expressed peptide backbone to said expressed enzyme, wherein said enzyme is heterologous to said peptide backbone.

16. A composition comprising an enzyme bound to a peptide backbone, wherein said composition is produced by a process comprising combining said peptide backbone with said enzymatic activity under conditions suitable to allow a non-covalent bond to form between said peptide backbone and said enzymatic activity, wherein said enzymatic activity is retained subsequent to said combination.

17. A method for producing a composition comprising an array of enzymes bound to a peptide backbone, said method comprising:
(a) expressing DNA encoding said peptide backbone in a microorganism having been transformed with DNA encoding said peptide backbone;
(b) expressing DNA encoding said enzyme in a microorganism having been transformed with DNA encoding said enzyme; and
(c) binding said expressed peptide backbone to said expressed enzyme, wherein said enzyme is heterologous to said peptide backbone.

18. The method according to claim 17, comprising at least two enzymes non-covalently bound to said peptide backbone.

19. The method according to claim 17, wherein said one or more enzyme comprises protease, cellulase, lipase, peroxidase, xylanase, oxidase, oxidoreductase, laccase, lactase, lyase, polygalacturonase, β-galactosidase, glucose isomerase, β-glucoamylase, α-amylase, NADH reductase or 2,5DKG reductase.

20. The method according to claim 17, wherein said peptide backbone comprises scaffoldin derived from a microorganism which produces a cellulosomal or amylosomal complex.

21. The method according to claim 20, wherein said scaffoldin is derived from Clostridium sp. and comprises at least one internal repeating element and at least one cellulose binding domain.

22. The method according to claim 17, wherein said enzyme is non-covalently bound to said peptide backbone by means of a dockerin region of said enzyme.

23. The method according to claim 22 wherein said dockerin region comprises a dockerin derived from Clostridium sp. or a derivative thereof capable of non-covalently binding to said peptide backbone.

24. The method according to claim 22 wherein said dockerin comprises an amino acid sequence according to the sequence of CelS or CelD as shown in FIG. 1.

25. The method according to claim 21, wherein said scaffoldin comprises CipA, CipB or CbpA.

26. The method according to claim 25, wherein said scaffoldin comprises an amino acid sequence described in FIG. 6.

27. The method according to claim 20, wherein said scaffoldin comprises a plurality of internal repeating elements.

28. The method according to claim 27, wherein said heterologous enzyme further comprises a dockerin region capable of binding with said scaffoldin protein.

29. The method according to claim 28, wherein said dockerin region forms a non-covalent bond with said internal repeating units.
